(19) United States
(12) Plant Patent Application Publication
(10) Pub. No.: US 2004/0148668 P1
(43) Pub. Date: Jul. 29, 2004

(54) HYBRID TEA ROSE PLANT NAMED 'RUIY5991'

(76) Inventor: Henk C.A. de Groot, De Kwakel (NL)

Correspondence Address: CHRISTIE, PARKER & HALE, LLP, 350 WEST COLORADO BOULEVARD, SUITE 500, PASADENA, CA, 91105, US

(21) Appl. No.: 10/437,875

(22) Filed: May 13, 2003

(30) Foreign Application Priority Data
May 21, 2002 (QZ) 2002/0775

(51) Int. Cl. A01H 5/00

(52) U.S. Cl. PLT/131

(57) ABSTRACT

A new and distinct Hybrid Tea Rose plant having attractive red and white bicolor flowers.

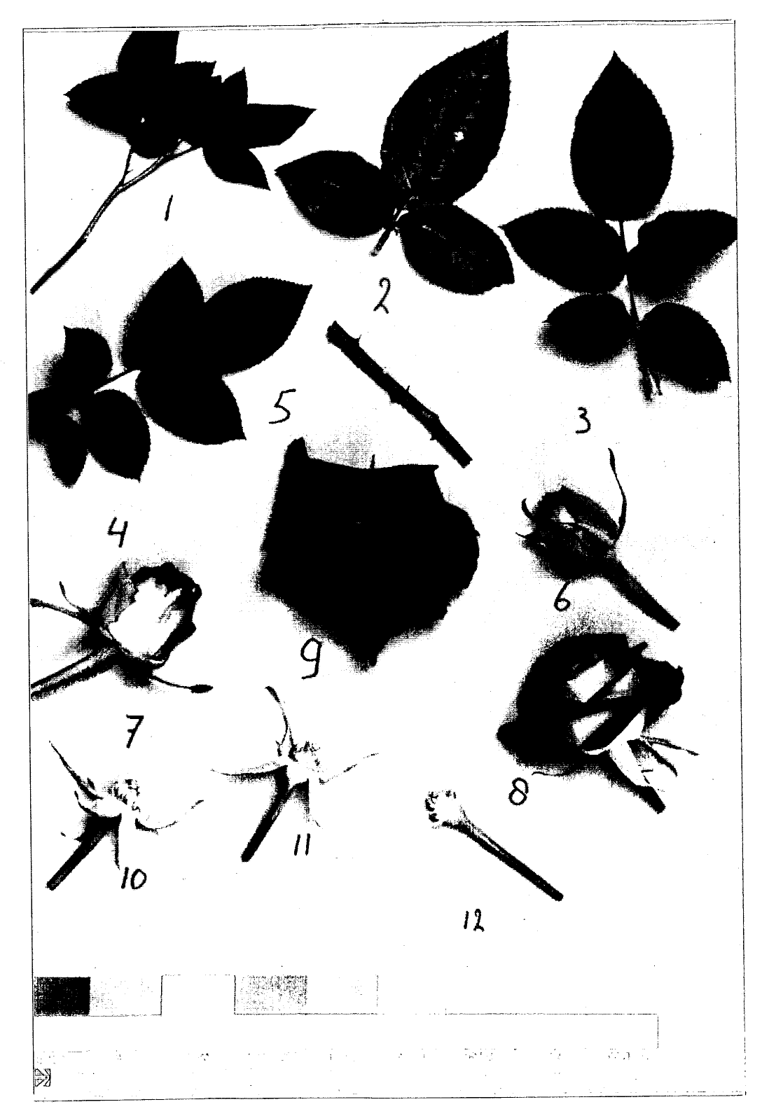

### BACKGROUND OF THE INVENTION

The present invention relates to a new and distinct variety of rose plant of the Hybrid Tea rose class, which was created by crossing as seed parent an unnamed, undistributed seedling and as a pollen parent the variety known as ‘RUIZON’. The varietal denomination of this new rose is named ‘RUIY5991’.

### SUMMARY OF THE INVENTION

Among the novel characteristics possessed by this new variety which distinguishes it from its parents and all other varieties of which I am aware are its attractive red and white bicolor flowers.

Asexual reproduction by propagation by grafting of the new variety as performed in De Kwakel, The Netherlands shows that the foregoing and other distinguishing characteristics come true to form and are established and transmitted through succeeding propagation.

### COMPARISON WITH PARENTS

Among other respects, the new variety differs from its parents in flower color, inasmuch as the seed parent has flowers that are red inside and white outside; and the pollen parent has red flowers, whereas the new variety has flowers that are red inside and white outside with the red color being brighter than the seed parent.

### BRIEF DESCRIPTION OF ILLUSTRATION

The accompanying illustration shows a typical specimen of the new variety depicted in color as nearly true as is reasonably possible to make the same in a color illustration of this character. In the illustration are shown: new growth 1; leaflets 2, 3 and 4; stem with thorns 5; flowers in different stages of development 6, 7, 8 and 9; and reproductive organs 10, 11 and 12.

### DESCRIPTION OF THE NEW VARIETY

The following is a detailed description of the new variety with color terminology in accordance with The Royal Horticultural Society Colour Chart (RHSCC). The terminology used in color description herein refers to plate numbers in the aforementioned color chart. Phenotypic expression may vary with light availability, environmental and cultural conditions.

The following observations are made of 9-month old plants grown in a greenhouse in De Kwakel, The Netherlands during January from a grafted cutting.

### FLOWER

Blooming habit: Continuous.

Bud.—Size: About 6 cm long and 5 cm wide Form: Broad ovate Color Outside: Near 62D Inside: Near 53A.

Sepals.—Size: Just before opening as long as bud Color: Upper Surface: Near 131A Undersurface: Near 137C, sometimes with leaf-like ends.

Peduncle.—Length: About 15 cm Aspect: Top about 5 cm. Is smooth; bottom hirsute Strength: Strong Color: Near 137C.

Bloom:

Size.—About 14 cm.

Borne.—Singly.

Form.—Side view of upper part is convex; side of view of lower part is concave.

Petalage.—About 36 petals.

Color (⅓ to ½ open).—Outer Petals Top Surface: Near 57A Undersurface: Near 49D Inner Petals: Top Surface: Near 57A Undersurface: Near 49D Base of Petals (point of attachment): Near 4C to 4D to 49D to 57A General tonality from a distance: Red and white bicolor.

Color (fully open):

Outer petals.—Top Surface: Near 57A Undersurface: Near 49D

Inner petals.—Top Surface: Near 57A Undersurface: Near 49D.

Base of petals (point of attachment).—Near 4C to 49D to 57A.

General tonality from a distance.—Red.

Color change.—None.

General tonality from a distance.—Red.

Petals:

Texture.—Smooth Appearance Inside: Velvety Outside: Velvety to satiny.

Form.—Round.

Arrangement.—Imbricated.

Petaloids in center.—Many, about 19.

Persistence.—Persistent.

Fragrance.—None.

Lasting quality.—On the plant: About 14 days In the vase: About 9 days.

### REPRODUCTIVE ORGANS

Stamens, filaments and anthers:

Arrangement.—Irregularly arranged around styles.

Color.—Stamens: Near 11C Filaments: Bottom near 10B; top near 41C Anthers: Near 11C.

Pollen.—Near 199C.

Styles.—Uneven length between; about 1 to 1.5 cm; thin.

Stigmas.—Near 13C.

Hips.—Shape: Urn-shaped Size: Length about 1 cm; width at top about 1 cm.

### PLANT

Form: Upright; long stems.

Growth: Vigorous.

Foliage: Compound 3, 5 and 7 leaflet leaves.

Size.—Leaflets; length about 10 cm; width about 6 cm.

Quantity.—Approximately 14 leaves per stem.

Color.—New foliage Upper Side: Near 181A Underside: Near 180C Mature foliage: Upper Side: Near 139A Underside: Near 147B.

Shape.—Elliptic.

Texture.—Upper Side: Glossy Underside: Matte.

Edge.—Serrated.

Serration.—Serrate.

Petiole:

Top surface.—Near 185B.

Underside.—Near 166B.

Stipules: About 1.5 cm; near 137B.

Auricle: Not present.

Rachis: Surface top is flat; underside is rounded with prickles.

Wood:

New shoots.—Color Bark: Near 145B Wood: Near 146C.

Old wood.—Color Bark: Near 147B Wood: Near 149D.

Flower stem.—Approximately 80 to 90 cm.

Thorns: On lower portion of stem about 3 to 4 thorns between internodes; on upper portion no prickles.

Quantity.—Many.

Form.—Small, deep concave.

Length.—Short length about 0.7 cm; basis width about 0.4 cm.

Color.—Young: About 175B Old: About 175A.

Position.—Irregular.

I claim:

1. A new and distinct hybrid tea rose plant of the variety substantially as shown and described:
